(19) United States
(12) Patent Application Publication
(10) Pub. No.: US 2025/0226061 A1
(43) Pub. Date: Jul. 10, 2025

(54) Retention Time Trajectory Matching For Peak Identification In Chromatographic Analysis

(71) Applicant: THE REGENTS OF THE UNIVERSITY OF MICHIGAN, Ann Arbor, MI (US)

(72) Inventors: Xudong FAN, Saline, MI (US); Wenzhe ZANG, Ann Arbor, MI (US); Ruchi SHARMA, Ann Arbor, MI (US); Maxwell Wei-Hao LI, Troy, MI (US)

(21) Appl. No.: 17/841,404

(22) Filed: Jun. 15, 2022

Related U.S. Application Data
(60) Provisional application No. 63/219,581, filed on Jul. 8, 2021.

(51) Int. Cl. G16C 20/30; G01N 30/86; G16C 20/64; G16C 20/80; G16C 20/90

(52) U.S. Cl. CPC G16C 20/30; G01N 30/8696; G16C 20/64; G16C 20/80; G16C 20/90

(57) ABSTRACT

Retention time drift caused by fluctuations in physical factors such as temperature ramping rate and carrier gas flow rate is ubiquitous in chromatographic measurements. Proper peak identification and alignment across different chromatograms is critical prior to any subsequent analysis. This work introduces a peak identification method called retention time trajectory (RTT) matching, which uses chromatographic retention times as the only input and identifies peaks associated with any subset of a predefined set of target compounds. RTT matching is also capable of reporting interferents. An RTT is a 2-dimensional (2D) curve formed uniquely by the retention times of the chromatographic peaks. The RTTs obtained from the chromatogram of a test sample and of pre-characterized library are matched and statistically compared. The best matched pair implies identification. Unlike most existing peak alignment methods, no mathematical warping or transformations are involved.

### CROSS-REFERENCE TO RELATED APPLICATION

This application claims the benefit of U.S. Provisional Application No. 63/219,581, filed on Jul. 8, 2021. The entire disclosure of the above application is incorporated herein by reference.

### GOVERNMENT CLAUSE

This invention was made with government support under R01 OH011082 awarded by the Centers for Disease Control and Prevention and FA8650-19-C-9101 awarded by the U.S. Air Force, Air Force Materiel Command. The government has certain rights in the invention.

### FIELD

The present disclosure relates to improved techniques for identifying analytes in a mixture.

### BACKGROUND

Gas chromatography (GC)-based volatile organic compound (VOC) analysis can be classified into untargeted analysis and targeted analysis. The former involves evaluation of chemical substances in an unknown sample, whereas the latter aims only at a predetermined list of interesting compounds or a subset of those, with all other VOCs treated as interferents. Due to the complexity of sample composition and the lack of pre-existing knowledge, accurate identification in untargeted analysis requires confirmation or cross-validation by at least two parameters, such as chromatographic retention time (RT) and mass spectrometry (MS) fragmentation profile. In contrast, in targeted analysis oftentimes only the retention time is used for compound identification in order to avoid using bulky and expensive mass spectrometry. Therefore, targeted analysis has broad applications in on-site real-time measurements, such as environmental protection, working place environment monitoring, industries (e.g., petroleum and food), and metabolomics.

For targeted analysis, the retention time of each peak in the GC chromatogram is compared with the pre-installed values of all compounds of interest in a library. In any given sample, a positive alarm is reported when the retention time of a peak matches a corresponding time in the library; the lack of any match instead means that a peak would be ignored or reported as an interferent. However, variations in physical factors such as ambient temperature, column temperature ramping profile, and carrier gas flow rate can affect the retention time of each peak from run to run, which hinders identification or triggers false alarms. The inability of correct peak identification with only retention times exacerbates when a sample contains a large number of compounds or some of the targeted peaks are closely eluted out in a chromatogram. Consequently, proper matching or alignment of chromatographic peaks across different samples is a crucial preprocessing step prior to any subsequent analysis.

A simple and popular solution is data binning, which divides the signals into bins (e.g., histogram) and incorporate all data into a recognition profile for each measurement. The binning method is easy to use and shows acceptable performance in processing both chromatogram and spectrum when the peak drift from sample to sample is much smaller than the distance between two adjacent peaks. However, in the presence of large peak drifts, this approach suffers from reduced resolution and information loss. The time warping technique, such as segment-wise correlation optimized warping (COW), point-wise dynamic time warping (DTW), global polynomial model-based parametric time warping (PTW), multiscale peak alignment (MSPA), and other variants is one of most commonly adopted methods to correct retention time drifts across chromatograms. It aligns a whole measured chromatogram profile against a reference chromatogram using pattern recognition routines in order to achieve peak identification. While time warping is powerful and works well with samples of various complexities, an accurate warping-based aligning demands fine tuning of alignment parameters that can often involves human intervention, thus making automated peak identification less reliable. Moreover, all warping-based methods can suffer, to different degrees, from misalignments and are concentration-sensitive, even with samples of the same compositions. In some cases, warping-based aligning approaches may not be able to yield exactly the same retention time value for the same analyte from different measurements. Consequently, subsequent RT value based peak identification or statistical analysis often require further value correction via data binning or clustering. Machine learning based aligning approaches, which utilize artificial intelligence systems to acquire knowledge by extracting patterns from data, are also able to achieve positive alignment with decent accuracy, and they are amenable to automation without relying on human intervention. However, these approaches often employ the mass spectra of the peaks as one the of key subnetworks among the overall network architecture, making it more suitable for bulky mass spectrometry based analysis rather than onsite monitoring. Also, similar to most machine learning based approaches, it suffers from high computational cost during parameter training and feature extraction.

While the aforementioned methods can mitigate the peak drift issue across chromatograms, they all suffer from a number of drawbacks, particularly relevant to targeted analysis, which makes them unsuitable for compound identification in targeted analysis. First and foremost, the total number of peaks in the chromatogram obtained from the test sample needs to be exactly the same as in the reference chromatogram. If only a subset, or a single species in an extreme case, of the compounds of interest (target compounds), are present in the sample chromatogram, which is often the case for targeted analysis (e.g., pipeline leakage detection in a chemical plant), chromatogram aligning fails, as the corresponding peaks have nothing to be aligned with. Second, foreign interferents cannot be filtered out. If an additional peak is present in a chromatogram, it would be treated as one of the target compounds (misalignment) and/or it may cause failure in alignment of the whole chromatogram profile. Consequently, there has been an unmet need for an MS-free and chromatogram-based peak identification method that is able to identify any arbitrary subset of the target compounds as well as to report interferents.

This disclosure proposes a retention time trajectory (RTT) matching method for peak identification. With peak retention times as the only input, RTT matching can identify peaks associated with any subset of target compounds as well as filter interferents outside of the targets.

This section provides background information related to the present disclosure which is not necessarily prior art.

### SUMMARY

This section provides a general summary of the disclosure, and is not a comprehensive disclosure of its full scope or all of its features.

A computer-implemented method is presented for identifying compounds in a mixture. A library of retention time trajectories are stored on a non-transitory computer readable medium, where each trajectory in the library of trajectories represents a set of retention times for a group of known compounds in a mixture. The method includes: receiving a set of retention times for analytes to be identified in a sample under test; creating a retention time trajectory for the sample under test based on correlation between retention times in the set of retention times and the group of known compounds; comparing retention time trajectory for the sample under test to the trajectories in the library of retention time trajectories; and identifying compounds in the sample under test based on the comparison of the retention time trajectory for the sample under test to trajectories in the library of retention time trajectories.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram depicting an example arrangement for a gas chromatograph.

FIG. 2A is a graph showing an example retention time trajectory from a chromatogram.

FIG. 2B is a graph showing an example library of retention time trajectories.

FIG. 3 is a flowchart depicting a technique for identifying analytes in a mixture using a chromatograph.

FIGS. 4A-4D illustrate identification of analytes, where the number of analytes in the sample under test is less than the number of target analytes.

FIGS. 5A-5C are graphs illustrating calculations of sum of squared residual and mean squared residual.

FIG. 6 is a diagram illustrating experimentally generated chromatograms.

Corresponding reference numerals indicate corresponding parts throughout the several views of the drawings.

### DETAILED DESCRIPTION

Example embodiments will now be described more fully with reference to the accompanying drawings.

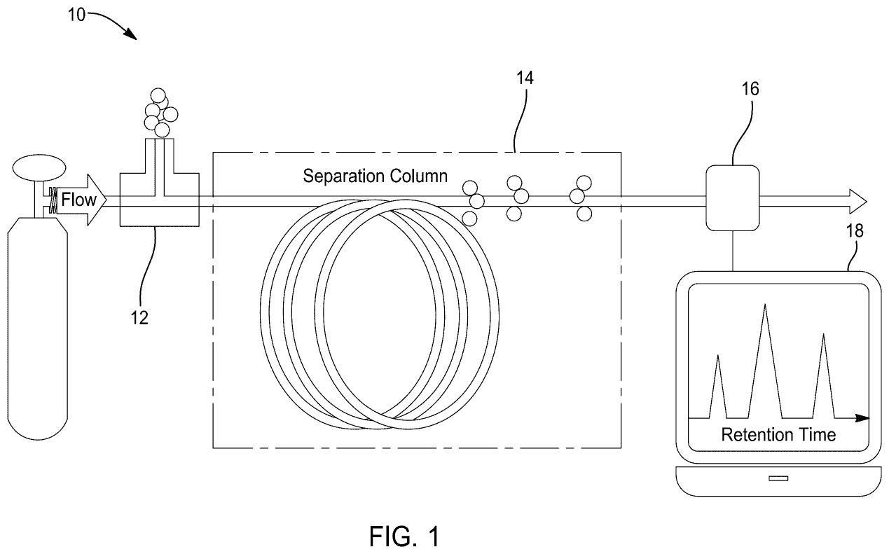

FIG. 1 depicts an example arrangement for a gas chromatograph 10. The gas chromatograph 10 is comprised of an injection port 12, at least one separation column 14, and a detector 16. The detector 16 is preferably interfaced with a computing device 18. In one example, the computing device 18 is a desktop computer and monitor although other types of computing devices fall within the scope of this disclosure. While reference is made throughout this disclosure to a gas chromatograph, the techniques presented herein are applicable to other types of chromatographs (e.g., liquid chromatography) as well.

During operation, the injection port 12 is configured to receive a sample and a carrier gas which form a mixture. It is understood that the sample may include one or more target compounds. The gas mixture flows through the separation column 14 and to the detector 16. The separation column 14 operates to separate analytes from one another as the mixture passes through. The detector 16 in turn receives the gas from the separation column 14 and quantitatively measures the analytes in the gas, such as a retention time for each analyte.

Between GC analytical runs, the retention time (RT) for a given compound may drift due to perturbation of various physical factors including ambient temperature, column temperature programing profile, and carrier gas flow rate. The influence of these perturbations on the analytes in a sample can be quite different due to their diverse characteristics (such as volatility, polarity, functional groups, etc.). Consequently, the retention time drifts of the analytes in a chromatogram are often non-linear and unpredictable. The retention time deviation (ΔRT) against retention time has been described using quadratic functions in PTW or local regression fitting (LOESS) in XCMS, which often over-simplifies the diverse and complex nature of retention time drifting. These methods are either limited to samples with the same constitutions or require MS-based peak matching before final aligning.

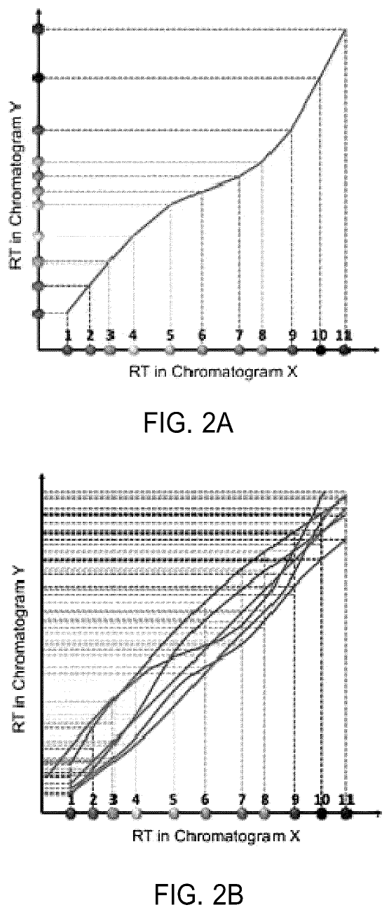

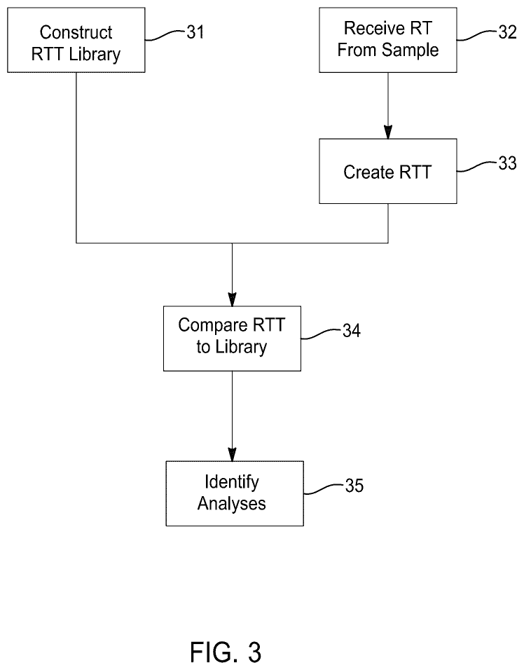

Retention time trajectories (RTT) are used for peak identification in this disclosure. With reference to FIG. 2A, a RTT is made up of a series of retention times of all compounds (peaks) in a chromatogram obtained under one set of experimental conditions (such as ambient temperature, column temperature ramping profile, and carrier gas flow rate, etc.) and uniquely represents one particular condition. The X-axis represents the retention times (RT_{X}) for a reference mixture, referred to as Chromatogram X. The colored dots along the X-axis represent different compounds and are numerically labelled as 1, 2, 3, . . . 11. Similarly, the Y-axis represents the retention time, RT_{Y}, for a sample under test, referred to as Chromatogram Y. The entire set of coordinates, (RT_{X,compound i}, RT_{Y,compound i}), where “compound i” refers to a specific compound, form a trajectory in 2D. In other words, to derive an RTT, each retention time in the set of retention times for a sample under test is paired in relation to a set of reference retention times, thereby creating a retention time trajectory.

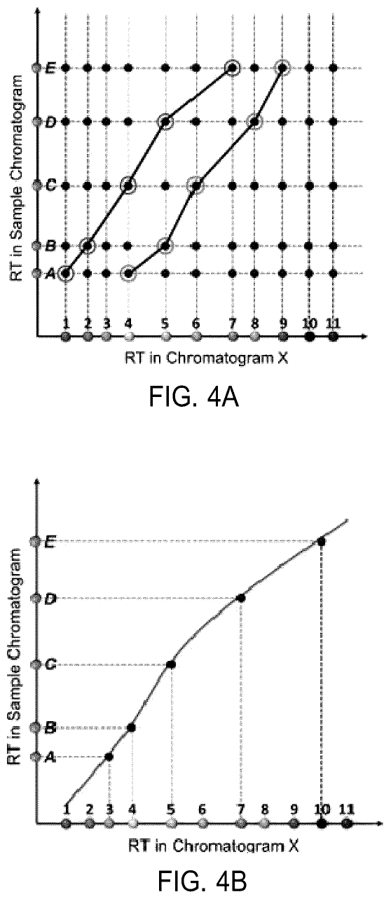

FIG. 3 illustrates an improved technique for identifying analytes in a mixture using a chromatograph. As a starting point, experimentally generate multiple chromatograms under various experimental conditions (temperature ramping profiles and flow rate, etc.) using a mixture that contains a group of target (known) compounds and internal standards (if needed) as indicated at 31. Retention times in each chromatogram are extracted and stored as a set of retention times, RTT_{lib}s, in a library of retention time trajectories on a non-transitory computer readable medium. Each trajectory corresponds uniquely to a chromatogram obtained under a certain set of conditions (temperature ramping and flow rate, etc.). Note that the chemical identities of all peaks in any RTT_{lib} are known. The sets of trajectories represent chromatograms under various experimental conditions (i.e., the library of RTT_{lib}s) as is shown in diagram in FIG. 2B. In this way, a library of retention time trajectories is constructed.

Construction of an RTTₛₐₘₚₗₑ for a sample under test is similar. Each RTTₛₐₘₚₗₑ is made up of a series of coordinates, (RT_{X,compound i}, RT_{sample,peak j}), where RT_{sample, peak j} refers to the retention time of a peak in the chromatogram obtained from the sample under test (i.e., sample chromatogram). Note that the peaks in the sample chromatogram may contain only a sub-set of target compounds as well as possible interferents. Since the chemical identities of the detected peaks are unknown before analysis, multiple RTTₛₐₘₚₗₑs may form for a given sample chromatogram, each of which corresponds to one set of peak identification results. The challenge is to eliminate all impossible RTTₛₐₘₚₗₑs and find the one that best matches one of the RTT_{lib}s in the library.

With continued reference to FIG. 3, a set of retention times for analytes in a sample under test are received at 32. A retention time trajectory is created for the sample under test at 33 by correlating the retention times in the set of retention times with retention times for the group of known compounds. In one embodiment, the retention time trajectory is created by pairing each retention time in the set of retention times in relation to a set of reference retention times for the group of target (known) known compounds. The retention time trajectory for the sample under test is then compared at 34 to the trajectories in the library of retention time trajectories. More specifically, a similarity measure is calculated between the retention time trajectory for the sample under test and each of the trajectories in the library of trajectories. In one embodiment, the similarity measure is further defined as a mean square residual. Other suitable comparison techniques may include but are not limited to linear regression, logistic regression, mean absolute differences and K nearest neighbors.

Based on the comparison of the retention time trajectory for the sample under test to trajectories in the library of retention time trajectories, analytes in the sample under test are identified at 35. For example, the chemical identities for the detected peaks are extracted from the RTTₛₐₘₚₗₑ that best matches one of the RTT_{lib}s (e.g., smallest mean square residual). The identified compounds in the sample under test are then reported, for example visually on a display of the computing device 18.

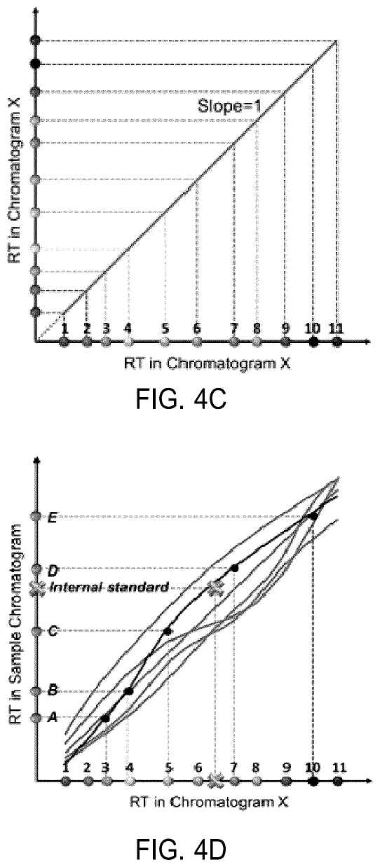

Consider a sample with no interferents present (i.e., all detected peaks are a subset of target compounds). Assume a total of N_{tgt} target compounds and Nₛₐₘₚₗₑ detected peaks in the sample under test. A matrix with N_{tgt}×Nₛₐₘₚₗₑ intersections (coordinates) is then formed in the 2D diagram (marked as black dots, i.e., coordinates, FIG. 4A), since each peak in the sample under test is unknown before final identification and in principle can be any of the N_{tgt} target compounds. Consequently, a total of C(N_{tgt}, Nₛₐₘₚₗₑ) sets of RTTₛₐₘₚₗₑs can be formed by connecting one black dot in each of the Nₛₐₘₚₗₑ rows, two of which are exemplified as black lines in FIG. 4A.

$$C ( {N}_{tgt} , {N}_{sample} ) = \frac{{N}_{tgt} !}{( {N}_{tgt} - {N}_{sample} ) ! {N}_{sample} !}$$

is a combinatorial number and can be an extremely large when N_{tgt} is above 20 and Nₛₐₘₚₗₑ is around half of N_{tgt} (e.g., C(20,10)=184756).

However, not all C(N_{tgt}, Nₛₐₘₚₗₑ) RTTₛₐₘₚₗₑs are possible and many need to be eliminated first before the comparison with the RTT_{lib}s in the library, which expedites computation and avoids false identifications. Example elimination rules are as follow.

First, one target compound can only be mapped to one peak in the sample chromatogram. Therefore, any RTTₛₐₘₚₗₑ with a vertical section between any two consecutive coordinates (or intersections) in the 2D diagram should be eliminated.

Second, the elution order should be preserved. Therefore, any RTTₛₐₘₚₗₑ with a section that has a negative slope between two consecutive coordinates (i.e., opposite elution order in library and sample chromatograms) should be eliminated.

Third, the retention time drifts arise from minor perturbation and the resulting deviations (ΔRT) should be small values within a certain range. Therefore, only coordinates falling within the cutoff range of RTₛₐₘₚₗₑ+Δt should be considered. The value of Δt can be estimated empirically. For example, sample chromatograms with larger drifts require sufficiently large Δt (e.g., larger than typical RT drifting range in the RTT library). Note that the RTT matching algorithm is still able to effectively identify the peaks even without applying this criterion, but a reasonable estimation of Δt significantly reduces the computational cost by narrowing down possible RTTₛₐₘₚₗₑs.

In this scenario (i.e., where number of retention times in the sample under test is less than number of compounds in the group of known compounds), possible trajectories for retention times in the set of retention times are enumerated In some embodiments, the number of possible trajectories is constrained, for example such that a given retention time in the set of retention times is mapped to only known compound and/or the order of retention times is maintained in accordance with the group of known compounds.

Once the set of possible trajectories, RTTₛₐₘₚₗₑs, are formed, each individual RTTₛₐₘₚₗₑ should be compared with all RTT_{lib}s to find the best matched RTTₛₐₘₚₗₑ. In other words, one need to find which set of coordinates in FIG. 4A fall on one RTT_{lib}. Assuming a total of n_{lib} RTT_{lib}s stored in the library and nₛₐₘₚₗₑ possible RTTₛₐₘₚₗₑs generated from the sample chromatogram, n_{lib}×nₛₐₘₚₗₑ pairs of RTT_{lib} and RTTₛₐₘₚₗₑ are formed and compared with each other. For example, by comparing the trajectories in FIG. 2B, two groups of black dots, which are composed of two different RTTₛₐₘₚₗₑs, fall simultaneously on two different RTT_{lib}s (FIGS. 4B and 4C), respectively, and yield different chemical identification results for Peaks C and D. These are either Compounds 5 and 7 (FIG. 4B), or 6 and 8 (FIG. 4C).

To circumvent this issue, one can introduce internal standard compounds (i.e., internal standards) outside of the list of target compounds to anchor the RTT_{lib}s. In both RTT_{lib} library preparation and actual measurement of the test sample, the internal standard(s) are spiked into the mixture containing all target compounds (for RTT_{lib} library preparation) or the sample under test. The peaks corresponding to these standards are identified during the data preprocessing and then used to generate the RTT along with all other peaks. As depicted in FIG. 4D, when an internal standard (marked with a cross) is introduced, only one of the two RTT_{lib}s can be anchored (blue line) and thus a single set of identification result is obtained.

Introduction of internal standard(s) can further increase identification accuracy and significantly expedite computation by narrowing down possible RTTₛₐₘₚₗₑs and RTT_{lib}s, since (1) all possible RTTₛₐₘₚₗₑs and all RTT_{lib}s must go through the coordinate(s) formed by the internal standard(s) and (2) since the elution order is preserved, the whole 2D diagram can be divided into small regions determined by the internal standards' coordinates and only the RTTₛₐₘₚₗₑs falling within these regions are possible candidates. When there is only a single analyte in the sample under test, identification of its corresponding peak in the chromatogram is nearly impossible without internal standard(s), as formation of an RTT requires at least two coordinates. The addition of one or more coordinates resulting from the internal standards allows for the creation of the RTTₛₐₘₚₗₑs for more accurate identification of the single peak. Misidentification can be significantly reduced even when the chromatogram-to-chromatogram retention time drift of the same compound is greater than the distance between two adjacent peaks, which has long been the bottleneck of many peak matching or profile aligning algorithms. In practice, internal standards can be strategically positioned in the region with more drastic variations to more effectively narrow down RTTₛₐₘₚₗₑ and RTT_{lib}s selection. Note, similar to all internal standards based chromatographic analysis methods, the addition of internal standards might potentially worsen the co-elution issues with the neighboring target compounds. To avoid this, internal standards whose RTs fall in the chromatogram sections with low peak densities are preferred.

Internal standards have commonly been utilized by many aligning algorithms, in which retention time drift is corrected by first dividing the chromatogram into multiple sections delineated by the standards, and then applying linear stretching/compressing in each section. However, these methods cannot account for the various non-linear drifts that often occur between any two standards. To make the linear stretching/compressing more accurate, more internal standards need to be introduced to reduce sections size to the point that linear approximation within each section is valid. This makes both sample preparation and peak identification much more complicated. More advanced techniques employ polynomial fitting within each section to account for non-linearity. However, polynomial fitting highly depends on experimental conditions and is required for each section in a chromatogram, thus hampering automation in peak identification. In contrast, this technique compares the retention times of the chromatogram (or its corresponding RTT) globally, which automatically takes into account any non-linearities within each section. To demonstrate the advantages of RTT matching, one can use the same sample and internal standards to compare the performance of the RTT matching approach and linear warping as well as correlation optimized warping.

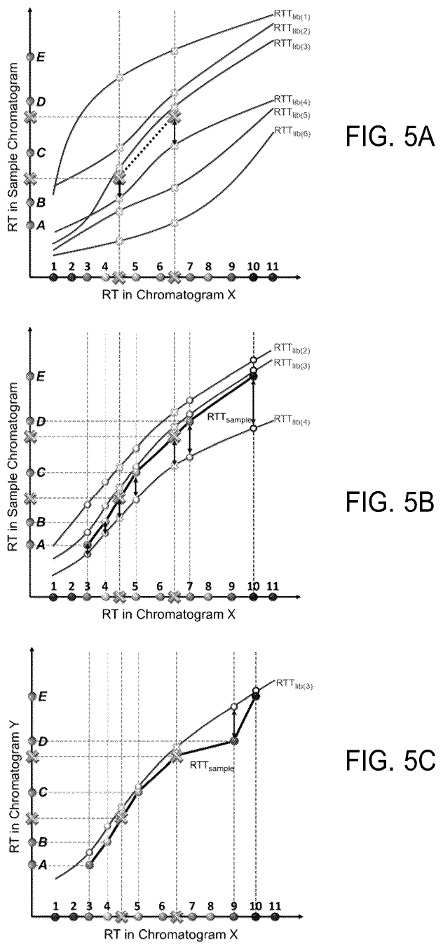

In an example embodiment, mean squared residual (MSR) is used to compare trajectories. While coordinates in the X-axis are discrete, their variation along the Y-axis is continuous due to continuously varying experimental conditions. In theory, the number of the RTT_{lib}s is infinite. In practice, only a limited number of conditions and hence a limited number of RTT_{lib}s can be characterized and stored. Consequently, while the RTTₛₐₘₚₗₑ for a sample under test may not exactly match any RTT_{lib} stored in the library, the most similar one can still be easily found. To globally compare similarities between an RTTₛₐₘₚₗₑ and an RTT_{lib}, calculate the mean squared residuals (MSR) of RTs from the same compounds between these two trajectories, as illustrated in FIG. 5A-5C. A smaller MSR indicates higher similarity between two trajectories, as exemplified in FIG. 5B where RTT_{lib(3)} is the most similar to the RTTₛₐₘₚₗₑ in the figure. The MSR is normalized (or scaled) from the sum of squared residuals (SSR) by the total number of paired compounds to ensure that it does not grow as the number of pairs grows. This is important when one needs to compare RTTₛₐₘₚₗₑs with different numbers of target compounds, for example, an RTTₛₐₘₚₗₑ of 6 target compounds is compared with an RTTₛₐₘₚₗₑ of 5 target compounds plus 1 interferent (the case of interferent identification will be discussed below).

In order to expedite the computation, it is not necessary to compare each RTTₛₐₘₚₗₑ with all RTT_{lib}s. Instead, first use internal standards RTs to anchor the best matching sets of RTT_{lib}s by calculating the SSR of internal standards (SSR^{std}), as shown in FIG. 5A. Since all possible RTTₛₐₘₚₗₑs must go through the coordinates formed by internal standards, they have the same SSR^{std} for a given RTT_{lib(i)}, where i refers to a specific pre-characterized RTT_{lib}. Assuming that there are a total of N_{std} internal standards, the SSR^{std} between any RTTₛₐₘₚₗₑ and an RTT_{lib(i)}, denoted as SSR_{lib(i)}^{std}, can be calculated as

$${SSR}_{lib ( i )}^{std} = {\sum }_{k = 1}^{{N}_{std}} {( {RT}_{lib ( i )}^{std ( k )} - {RT}_{sample}^{std ( k )} )}^{2} , \qquad ( 1 )$$

where RT_{lib(i)}^{std(k)} is the retention time of one internal standard, k, in RTT_{lib (i)}, and RTₛₐₘₚₗₑ^{std(k)} is the retention time of the same internal standard in the sample chromatogram. The SSR^{std} of all RTT_{lib}s are sorted out ascendingly with the top ones, which have the least SSR^{std}s, giving the potentially matched RTT_{lib}s. All other RTT_{lib}s in the library, which have higher SSR^{std}s, can be eliminated.

Next, the RTTₛₐₘₚₗₑ and RTT_{lib} are further compared based on retention times of both internal standards and target compounds by sorting the MSR (see, FIG. 5B). In this step, only the top RTT_{lib}s (e.g., the first half or the top 20 RTT_{lib}s) with the lowest SSR^{std}s are selected. Assuming that, in addition to N_{std} internal standards, there are Nₛₐₘₚₗₑ peaks to be identified in the test sample, the MSR between one RTT_{lib} (denoted as RTT_{lib(i)} and one RTTₛₐₘₚₗₑ (denoted as RTTₛₐₘₚₗₑ₍ⱼ₎ can be calculated as

$$MSR = \frac{{SSR}_{lib ( i ) , sample ( j )}}{{N}_{std} + {N}_{sample}} = ( {SSR}_{lib ( i )}^{std} + {\sum }_{l = 1}^{{N}_{sample}} {( {RT}_{lib ( i )}^{compound ( l )} - {RT}_{sample ( j )}^{compound ( l )} )}^{2} ) / ( {N}_{std} + {N}_{sample ) ,} \qquad ( 2 )$$

where RTₛₐₘₚₗₑ₍ⱼ₎^{compound(l)} is the retention time of the Compound l in RTT_{lib(i)}, and RTₛₐₘₚₗₑ₍ᵢ₎^{compound (l)} is the retention time of a peak in the sample chromatogram that is hypothetically assigned to the same compound (i.e., Compound l) in RTTₛₐₘₚₗₑ₍ⱼ₎. Note that all RTTₛₐₘₚₗₑs have the same retention time for each peak, but each peak can hypothetically be paired with a different compound in different RTTₛₐₘₚₗₑ, which has been discussed previously (e.g., FIG. 4A). The MSR of all RTT_{lib}s are sorted out ascendingly. The first in the list has the minimum MSR value denoted as MSR_{sample(j),min}, which is generated by the RTT_{lib} that best matches RTT_{sample (j)}.

Since each RTTₛₐₘₚₗₑ is formed by pairing detected peaks with one set of target compounds, it represents one set of peak identification results. For any RTTₛₐₘₚₗₑ₍ⱼ₎, the best matched RTT_{lib} can be found by screening all RTT_{lib}s in the library and finding the one that generates MSR_{sample(j),min}. If there is another RTTₛₐₘₚₗₑ, denoted as RTTₛₐₘₚₗₑ₍ⱼ₎, that has MSR_{sample(j),min} smaller than MSR_{sample(j), min}, it means that RTTₛₐₘₚₗₑ₍ⱼ₎ better matches one of the RTT_{lib}s in the library. Therefore, corresponding identification results from RTTₛₐₘₚₗₑ₍ⱼ₎ are more possible than those from RTTₛₐₘₚₗₑ₍ⱼ₎.

In targeted analysis, interferents are the compounds not on the list of target compounds and need to be filtered out. In the proposed algorithm, two criteria are used to identify the presence of interferents. First, for a particular peak in the sample chromatogram, if none of the retention time values in the RTT_{lib}s falls in the range of RTₚₑₐₖ±Δt, this peak is identified as an interferent. In other words, the presence of an interferent in the target sample is identified when a given retention time in the sample under test falling outside a tolerance for a corresponding retention time in the library of trajectories. The value of Δt can be chosen empirically and can be set higher than a typical retention time drift range. Second, for one particular pair of RTTₛₐₘₚₗₑ₍ᵢ₎ and RTT_{lib(j)}, if the squared residual between one peak (for example, Peak D in FIG. 5C) in RTTₛₐₘₚₗₑ₍ᵢ₎ and its paired compound (Compound 9 in FIG. 5C) in RTT_{lib(j)} is much larger (e.g., twice) than MSR_{lib(i), sample (j)}, it is highly likely that this peak is an interferent. The validity of this approach lies in the fact that all other coordinates formed by the detected peaks and their paired target compounds well match RTT_{lib(j)}, except for the one formed by Peak D paired with Compound 9. A new MSR_{lib(i),sample (j)} is then calculated by normalizing SSR_{lib(i),sample (j)} which excludes residuals from all identified interferents, with N_{std}+Nₛₐₘₚₗₑ−N_{interf} (N_{interf} is the total number of identified interferents). Based on this, all possible peak identification results, with or without interferents, can be ranked by MSRs. The results with the smallest MSR give the highest confidence level.

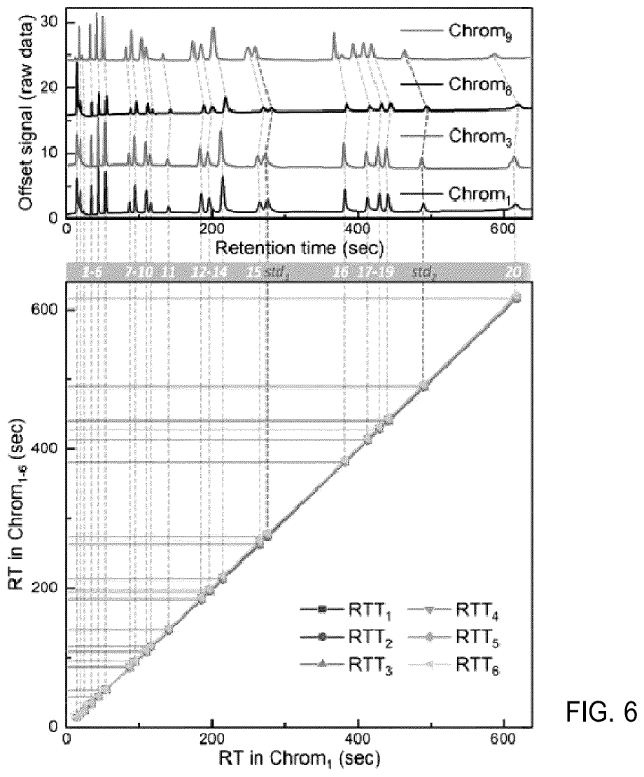

The retention time trajectory matching approach is validated using nine chromatograms obtained with NovaTest P300 GC provided by Nanova Environmental, Inc., which is equipped with a 6 m long Rtx-VMS column (Restek, Bellefonte, PA, USA) and a microfluidic photoionization detector. The chromatograms were generated under the same nominal experimental setting (carrier gas: helium; flow rate: 3.5 mL/min; temperature programming profile: 40° C. held for 5 min, ramped to 70° C. at 30° C./min, held for 2 min, then ramped to 150° C. at 30° C./min, and held for 1 min). The injected mixture is part of EPA Method TO-14. Exemplary chromatograms are presented in FIG. 6.

Detection of a peak in a chromatogram is accomplished by scanning for local maxima and the associated peak apex positions (i.e., retention times). A series of retention times are extracted, which are used to form the RTT_{lib}s or RTTₛₐₘₚₗₑs. Therefore, the cumbersome chromatographic data (i.e., a large 2D array of detection signals) are converted to a simple list of retention times, which significantly reduces data storage and processing workload. Extensive preprocessing (e.g., baseline removal) and broad background variations can also be eliminated since only the local maxima (i.e., peak apexes) are extracted.

Out of nine experimentally generated chromatograms, six chromatograms (denoted as Chrom₁₋₆) are used in the library, forming RTT_{lib}s. The remaining three (denoted as Chrom₇₋₉) are used to generate tests to validate the approach in various scenarios.

There are a total of 22 peaks in each measured chromatogram, among which 20 are treated as target compounds and the other two are used as the internal standards. The retention times and compound IDs of Chrom₁ are summarized in table below.

| Retention Time (sec) | Compound ID | Compound Name |
|---|---|---|
| 13.9 | 1 | Unknown |
| 19 | 2 | 1,1-Dichloroethene |
| 23.7 | 3 | Unknown |
| 33.6 | 4 | cis-1,2-Dichloroethene |
| 43.9 | 5 | Benzene |
| 53.6 | 6 | Trichloroethylene |
| 86.2 | 7 | cis-1,3-Dichloropropene |
| 94.4 | 8 | Toluene |
| 109.2 | 9 | Tetrachloroethylene |
| 115.9 | 10 | trans-1,3-Dichloropropene |
| 140.2 | 11 | 1,2-Dibromoethane |
| 184.8 | 12 | Chlorobenzene |
| 196.5 | 13 | Ethylbenzene |
| 214.4 | 14 | m,p-Xylene |
| 265.2 | 15 | o-Xylene |
| 275.8 | std₁ | Styrene |
| 382.1 | 16 | 1,3,5-Trimethylbenzene |
| 413.4 | 17 | 1,2,4-Trimethylbenzene |
| 429.3 | 18 | 1,3-Dichlorobenzene |
| 441.8 | 19 | 1,4-Dichlorobenzene |
| 490.5 | std₂ | 1,2-Dichlorobenzene |
| 617.6 | 20 | Hexachloro-1,3-Butadiene |

The RT deviation (ΔRT) against the RT in Chrom₁ for all chromatograms (Chrom₁₋₉) is plotted, showing strong non-linear drifting behavior. Note that while the chemical names for most compounds are known, the chemical identities of the first and the third eluted peaks are unknown (which might result from contamination and are only designated as ID 1 and ID 3, respectively. Nevertheless, the results presented in this disclosure remain the same regardless of whether the chemical names of those compounds are known.

In total, 3×Σᵢ₌₁²⁰[C(20, i)]=3.15×10⁵ validation tests are generated from Chrom₇₋₉, covering all subsets of the 20 target compounds (ranging from single compounds to 20 compounds). Moreover, three additional validation tests are generated, representing samples with a subset of target compounds and interferent(s). In all validation tests, peak identifications achieve 100% accuracy. Among all the validation tests, detailed results of 11 representative tests are presented, covering various MS-free chromatographic analysis scenarios.

In Scenario 1, different levels of retention time (RT) drift. The retention time drift is within the RT drift range of the pre-characterized RTTs (see Tests 1, 2, 3 with Chrom₇ in Table S1). The retention time drift is slightly out of the RT drift range of the pre-characterized RTTs (see Tests 4, 5, 6 with Chrom₈ in Table 1B). The retention time drift deviates far from the RT drift range of the pre-characterized RTTs (see Tests 10 and 11 with Chrom9 in Table S3)

In Scenario 2, various sample components with different numbers of target compounds is tested. See Tests 1-6, 10 and 11 (out of a total of 20 target compounds, the target compound number in each test is 11, 13, 5, 9, 5, and 1, respectively).

In Scenario 3, sample under test incudes with or without interferents. Tests 7, 8, and 9 (Table S2) respectively represents samples of 5 target compounds plus a single interferent (interferent RT is far from neighboring target compounds), 5 target compounds plus single interferent (interferent is close to neighboring target compounds), and 9 target compounds plus 2 interferents. The remaining tests represent samples containing a subset of target compounds.

To validate the identification technique, first discuss the scenarios in which no interferents are present and all the detected peaks are a subset of the target compounds. Based on the retention times in Chrom₇ and Chrom₈, six groups of retention times are generated, three from Chrom₇ and three from Chrom₈, for six tests. These represent six different mixtures containing various subsets of the target compounds along with two internal standards (std₁ and std₂). Note that the retention time deviation in Chrom₇ is within the range of the retention time deviations in the chromatograms (Chrom₁₋₆) stored in the library, whereas the retention time deviation in Chrom₈ is slightly out of this range. For each test, four best peak identification results are enumerated based on the MSR (Table S1). The peaks in all six tests are successfully identified with the top result (i.e., smallest MSR) producing 100% accuracy. The 2nd-4th best results in each test also correctly identify most of the peaks with the best ones giving 100% accuracy and the worst ones misidentifying only one peak (marked with an asterisk “*”). Note that even a single-species sample (Compound 7 in Test 6) can be correctly identified due to the use of internal standards, despite it being very close to neighboring Compound 8. This is impossible for all warping-based chromatogram aligning approaches, since the peak has nothing to be aligned with.

Another three validation experiments (Tests 7-9 in Table S2) are generated based on the retention times in Chrom₈, which mimics scenarios in which both a subset of target analytes and interferents are present. In Tests 7 and 8, one hypothetical interferent peak is added at 340 s and 449 s, respectively. In particular, the added peak at 449 s is very close to the target Compound 19. In both cases, the top identification result successfully identifies all target compounds and singles out interferent related peaks with 100% accuracy. In Test 9, two hypothetical interferent peaks are added at 62 s and 395 s, respectively, which are very close to target Compounds 6 and 16. Both interferents and all target compounds are correctly identified. Like all other RT based peak identification methods discussed previously, the RTT matching based interferent identification works only when RT_{interferent} is sufficiently different from those of target compounds. If RT_{interferent} is the same as or extremely close to any target compound, the interferent cannot be identified. Additionally, the majority of the peaks in the sample should be the target compounds. If most peaks are interferents and only a few target compounds are present, the validity of this method may decrease, since the residuals of most peaks are very large. To circumvent these issues, one way is to further enrich the RTT library, either experimentally or through RTT hybridization. Introducing more internal standards might also be necessary.

It is also worth noting that the proposed RTT matching approach is intended for scenarios where RT drifts result from only minor fluctuations in experimental conditions and therefore the elution order is expected to hold among the measurements. When the experimental condition varies drastically (e.g., major changes in the device settings or ambient temperatures), the elution order may vary. Therefore, a new RTT library needs to be constructed under the new experimental conditions to avoid misalignment/misidentification in the RTT matching approach.

The above two issues (i.e., serious co-elution and elution order change) have always been the bottleneck for all existing MS-free chromatogram aligning algorithms. MS (and other spectroscopic methods such as infrared absorption spectroscopy) would potentially be needed for peak identification in these cases.

In one aspect of this disclosure, an ideal library of retention time trajectories should contain all possible RTT_{lib}s that cover all possible drift inducing conditions. If the library has only a limited number of RTT_{lib}s and when the sample chromatogram drift (or retention time deviation) exceeds the retention time deviations covered by the RTT_{lib}s, peak misidentification may occur, as exemplified by Tests 10 and 11 in Table S3. One method to enrich RTT_{lib}s is to experimentally generate as many RTT_{lib}s as possible by varying the experimental conditions around the nominal conditions. However, this is extremely labor intensive and difficult to realize. Alternatively, new RTT_{lib}s can be generated by linearly hybridizing existing experimentally obtained RTT_{lib}s. This method is valid because the retention time drift is caused by minor fluctuations of system physical factors and such a small perturbation in the retention time of one particular compound from one state (one RT) to another state (another RT) can be simplified as linear variation. RTT linear hybridization can be done using two, three, or more existing experimentally obtained RTT_{lib}s, i.e.,
C₁×RT_{lib(a)}^{compound(i)}+C₂×RT_{lib(b)}^{compound(i)}, or
C₁×RT_{lib(a)}^{compound(i)}+C₂×RT_{lib(b)}^{compound(i)}+C₃×RT_{lib(c)}^{compound(i)} where C₁, C₂, and C₃ are the linear coefficients, and RT_{lib(a)}^{compound(i)} refers to the retention time for Compound i in one of the RTT_{lib}s (i.e., RTT_{lib(a)}). The hybridization can easily generate more RTT_{lib}s of the intermediate states that may be difficult to obtain experimentally (due to either time limitations and/or difficulty in realizing the exact experimental conditions), which significantly increases the tolerance to more serious retention time drifts. Note that the retention time variation of one particular compound from one state to another is simplified to be linear, although ΔRT against retention time in one chromatogram is generally non-linear.

To validate the hybridization method, this disclosure uses two-RTT hybridization based on following three formulas:

$$( {RT}_{lib ( a )}^{compound ( i )} + {RT}_{lib ( b )}^{compound ( i )} ) / 2 , \qquad ( 1 )$$

$$2 {RT}_{lib ( a )}^{compound ( i )} - {RT}_{lib ( b )}^{compound ( i )} , and \qquad ( 2 )$$

$$2 {RT}_{lib ( b )}^{compound ( i )} - {RT}_{lib ( a )}^{compound ( i )} \qquad ( 3 )$$

to enrich the RTT_{lib}s in the library, where RTT_{lib(a)} and RTT_{lib(b)} are from Chrom₁₋₆ that are experimentally generated. Two tests (Tests 10 and 11) are generated based on Chrom₉, which has much more serious drift compared to Chrom₇₋₈ and any pre-characterized chromatograms (Chrom₁₋₆) in the library. As shown in Table S3, when the RTT library contains only the experimentally generated RTT_{lib}s, it fails to correctly identify all peaks in either Test 9 or 10. In contrast, with more RTT_{lib}s added through hybridization, all peaks are successfully identified with 100% accuracy. The accuracy of the 2nd-4th identification results in both tests are also increased.

In order to compare peak identification performance with other chromatogram aligning approaches, part of the above validation tests are also performed with whole chromatogram based COW method and two peak-list-based aligning methods, namely internal standard based linear warping approach and fast PTW²¹. Herein, Chrom₁ (or its corresponding peak list) is treated as the reference to be aligned with.

Tests 5 and 7 (based on Chrom₈), which respectively represent a sample without and with an interferent are used to evaluate COW and linear warping. The retention times after COW or linear warping are summarized in Tables S4 and S5, respectively. Note that in the reference chromatogram (i.e., Chrom₁), all the peaks are present, including all target compounds and internal standards. The sample chromatograms, which are to be aligned and identified, are reconstructed from Chrom₈, but contain only the peaks listed in Tests 5 and 7. The remaining peaks in Chrom₈ are replaced with the baseline.

The COW aligning results can be highly parameter dependent. When the peak compositions in the reference and sample chromatograms are same, optimal parameters can be easily chosen so that the apex position of the peak of the same elution order coincides between the sample chromatogram and the reference chromatogram. However, in the presence of only a subset (as in Tests 5 and 7), the peak in the sample chromatogram has no specific target peak to align to, and therefore, the COW alignment parameter selection becomes dubious. Similarly, the interferent peak (as in Test 7) might be misaligned to one of the target compound peaks in the reference chromatogram. Multiple COW alignments with various tuning parameters (slack and correlation power) were conducted. The identification results based on the RTs extracted from the aligned Chromₛₐₘₚₗₑ are summarized in Table S4. Out of the 7 peaks in Test 5, the best COW aligning only correctly identifies 5 peaks (slack=4 and correlation power=3) and the worst aligning fails to align any of the peaks (slack=1 and correlation power=1; slack=2 and correlation power=2). For Test 7, the highest identification accuracy reaches only 50% (slack=4 and correlation power=3) and the worst aligning fails with the whole chromatogram (slack=1 and correlation power=1; slack=2 and correlation power=2).

For the internal standard based linear warping method (Table S5), the same internal standards (std₁ and std₂) are employed. After alignment, none of the target compound associated peaks yields the same RT as in the reference chromatogram (Chrom₁). Therefore, peak identification for target compounds fails when RT values are compared (all peaks are identified as interferents, except the interferent peak itself).

Additionally, compare the peak identification performance of fast PTW algorithm, which is a further development of the original PTW²⁰ and the algorithm input is chromatogram peak list (retention times and peak heights). The peak listed in Chrom₁, which is treated as the reference, and Chrom_{8,9}, which are used to generate Tests 5, 7, 10 and, 11. Although misalignments are greatly reduced after fast PTW aligning, sub-second to seconds of RT difference for the same compound persists. Tests 5, 10, and 11 represent samples with only a subset of target compounds; Tests 7 represents a sample with a subset of target compounds and an interferent. The fast PTW fails in all four tests to identify correct peaks. In comparison with the reference, the RT differences of the same compound in the warped sample peak list fall in the range of sub-seconds (which could be solved by data clustering) to hundreds of seconds (which completely fails in peak identification). Note that the warped RTs of some peaks give an unreasonable negative values (e.g., Compound 1 in Test 10 with order=2, and Compound 17 in Test 10 with order=3).

Two publicly available liquid chromatography (LC) fruit metabolomics datasets from the Metabolights repository (http://www.ebi.ac.uk/metabolights) with identifiers MTBLS99 and MTBLS85 are used to generate additional validation tests. This demonstrates the application of the RTT matching algorithm to complicated samples and other chromatographic techniques. The first dataset consists of LC measurements of a pooled sample that was injected regularly as a quality control during the measurements of apple extracts. The second datasets are from LC measurements of carotenoids in grape samples. In all of the designed validation tests, all target compounds associated peaks are correctly identified.

The retention time trajectory approach has the following main features. First and foremost, the matching is conducted between the entire trajectories (RTTₛₐₘₚₗₑ and RTT_{lib}) rather than between the individual peaks in the sample and the reference chromatogram. Second, simple statistics is used, which avoids time consuming training or feature extraction used in machine learning. In one example, MSR is adopted to describe the similarities between RTTs, which works well with the chromatograms obtained in this article. Other statistical approaches, such as linear regression, can also be introduced within the framework of RTT matching. Third, hybridization of RTT_{lib}s greatly enriches the RTT library, which not only increases the tolerance to more serious drifting, but also significantly reduces the cost for RTT_{lib} generation through actual experimentations. In this disclosure, each hybridized RTT_{lib} is generated out of two experimentally obtained RTT_{lib}s. Fourth, the only input variables are the retention times of each peak instead of the whole peak profile or mass spectra, making the RTT approach insensitive to concentration or background, and eliminating the need for bulky instruments such as mass spectroscopy. All of the above features make RTT matching highly amenable to automation with low computation cost. Additionally, the method described here can be easily translated to other chromatographic techniques (e.g., ion exchange chromatography) and has great potential to be applied to other spectral data (e.g., nuclear magnetic resonance spectroscopy). Finally, the introduction of internal standards, though contributing to increasing identification accuracy and computation efficiency, are not always necessary. When an increased number of target compounds are present in the sample, the contribution of standard(s) becomes less prominent. To validate this, 10 of the validation test examples in the above discussions, except the single-species sample in Test 6, have been re-performed with RTT matching without use of any internal standard. All the peaks can be correctly identified.

Finally, it is worth noting that the applications of the RTT matching approach are not limited to just peak identification. For visualization purposes, chromatogram aligning can easily be achieved with the RTT matching approach. Briefly, one can choose any RTT_{lib} as the reference chromatogram to extract RTs of target compounds. Each peak in the sample chromatogram is identified via RTT matching approach. Peak profile can be fitted by the exponentially modified Gaussian (EMG) model with apexes shifted to the positions as in the reference. Aligned sample chromatograms can be formed by the summation of individual EMG reconstructed peaks.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

### APPENDIX

TABLE S1
Test data generated from Chrom₇
Test 1

| Retention time (sec) | | | 13.8 | 19 | 33.5 | 43.7 | 85.6 | 108.5 | 115 | 183.6 | 213.3 | 412.7 | 616 |
|---|---|---|---|---|---|---|---|---|---|---|---|---|---|
| Compound ID | | | 1 | 2 | 4 | 5 | 7 | 9 | 10 | 12 | 14 | 17 | 20 |
| Ranking | MSR | Accuracy | Individual peak identification result | | | | | | | | | | |
| 1^{st} | 0.57 | 100% | 1 | 2 | 4 | 5 | 7 | 9 | 10 | 12 | 14 | 17 | 20 |
| 2^{nd} | 0.71 | 100% | 1 | 2 | 4 | 5 | 7 | 9 | 10 | 12 | 14 | 17 | 20 |
| 3^{rd} | 0.91 | 100% | 1 | 2 | 4 | 5 | 7 | 9 | 10 | 12 | 14 | 17 | 20 |
| 4^{th} | 2.2 | 90.9% | 1 | 3* | 4 | 5 | 7 | 9 | 10 | 12 | 14 | 17 | 20 |

Test 2

| Retention time (sec) | | | 19 | 33.5 | 43.7 | 85.6 | 93.8 | 108.5 | 115 | 139.3 | 195.2 | 213.3 | 264 | 381.3 | 412.7 |
|---|---|---|---|---|---|---|---|---|---|---|---|---|---|---|---|
| Compound ID | | | 2 | 4 | 5 | 7 | 8 | 9 | 10 | 11 | 13 | 14 | 15 | 16 | 17 |
| Ranking | MSR | Accuracy | Individual peak identification result | | | | | | | | | | | | |
| 1^{st} | 0.67 | 100% | 2 | 4 | 5 | 7 | 8 | 9 | 10 | 11 | 13 | 14 | 15 | 16 | 17 |
| 2^{nd} | 0.74 | 100% | 2 | 4 | 5 | 7 | 8 | 9 | 10 | 11 | 13 | 14 | 15 | 16 | 17 |
| 3^{rd} | 1.14 | 100% | 2 | 4 | 5 | 7 | 8 | 9 | 10 | 11 | 13 | 14 | 15 | 16 | 17 |
| 4^{th} | 2.15 | 92.3% | 3* | 4 | 5 | 7 | 8 | 9 | 10 | 11 | 13 | 14 | 15 | 16 | 17 |

Test 3

| Retention time (sec) | | | | 85.6 | 108.5 | 195.2 | 381.3 | 428.2 |
|---|---|---|---|---|---|---|---|---|
| Compound ID | | | | 7 | 9 | 13 | 16 | 18 |
| | Ranking | MSR | Accuracy | Individual peak identification result | | | | |
| | 1^{st} | 0.88 | 100% | 7 | 9 | 13 | 16 | 18 |
| | 2^{nd} | 0.94 | 100% | 7 | 9 | 13 | 16 | 18 |
| | 3^{rd} | 1.48 | 100% | 7 | 9 | 13 | 16 | 18 |
| | 4^{th} | 5.74 | 100% | 7 | 9 | 13 | 16 | 18 |

Test data generated from Chrom₈
Test 4

| Retention time (sec) | | | 34.1 | 44.6 | 96.2 | 111.4 | 142.9 | 188.4 | 218.6 | 384.8 | 432.6 |
|---|---|---|---|---|---|---|---|---|---|---|---|
| Compound ID | | | 4 | 5 | 8 | 9 | 11 | 12 | 14 | 16 | 18 |
| Ranking | MSR | Accuracy | Individual peak identification result | | | | | | | | |
| 1^{st} | 2.42 | 100% | 4 | 5 | 8 | 9 | 11 | 12 | 14 | 16 | 18 |
| 2^{nd} | 2.86 | 100% | 4 | 5 | 8 | 9 | 11 | 12 | 14 | 16 | 18 |
| 3^{rd} | 3.34 | 100% | 4 | 5 | 8 | 9 | 11 | 12 | 14 | 16 | 18 |
| 4^{th} | 5.06 | 88.9% | 4 | 5 | 8 | 10* | 11 | 12 | 14 | 16 | 18 |

Test 5

| Retention Time (sec) | | | | 87.9 | 111.4 | 218.6 | 384.8 | 432.6 |
|---|---|---|---|---|---|---|---|---|
| Compound ID | | | | 7 | 9 | 14 | 16 | 18 |
| | Ranking | MSR | Accuracy | Individual peak identification result | | | | |
| | 1^{st} | 2.99 | 100% | 7 | 9 | 14 | 16 | 18 |
| | 2^{nd} | 3.74 | 100% | 7 | 9 | 14 | 16 | 18 |
| | 3^{rd} | 4.28 | 100% | 7 | 9 | 14 | 16 | 18 |
| | 4^{th} | 7.14 | 80% | 7 | 10* | 14 | 16 | 18 |

Test 6

| Retention Time (sec) | | | | 87.9 |
|---|---|---|---|---|
| Compound ID | | | | 7 |
| | | | | Individual peak |
| | Ranking | MSR | Accuracy | identification result |
| | 1^{st} | 3.80 | 100% | 7 |
| | 2^{nd} | 5.10 | 100% | 7 |
| | 3^{rd} | 5.80 | 100% | 7 |
| | 4^{th} | 16.75 | 100% | 7 |

TABLE S2
est data generated from Chrom₈
Test 7

| Retention time (sec) | | | 87.9 | 111.4 | 218.6 | 340 | 384.8 | 432.6 |
|---|---|---|---|---|---|---|---|---|
| Compound ID | | | 7 | 9 | 14 | Interferent | 16 | 18 |
| Ranking | MSR | Accuracy | Individual peak identification result | | | | | |
| 1^{st} | 2.99 | 100% | 7 | 9 | 14 | Interferent | 16 | 18 |
| 2^{nd} | 3.74 | 100% | 7 | 9 | 14 | Interferent | 16 | 18 |
| 3^{rd} | 4.28 | 100% | 7 | 9 | 14 | Interferent | 16 | 18 |
| 4^{th} | 7.14 | 83.3% | 7 | 10* | 14 | Interferent | 16 | 18 |

Test 8

| Retention time (sec) | | | 87.9 | 111.4 | 218.6 | 384.8 | 432.6 | 449 |
|---|---|---|---|---|---|---|---|---|
| Compound ID | | | 7 | 9 | 14 | 16 | 18 | Interferent |
| Ranking | MSR | Accuracy | Individual peak identification result | | | | | |
| 1^{st} | 2.99 | 100% | 7 | 9 | 14 | 16 | 18 | Interferent |
| 2^{nd} | 3.74 | 100% | 7 | 9 | 14 | 16 | 18 | Interferent |
| 3^{rd} | 4.11 | 83.3% | 7 | 9 | 14 | 16 | 18 | 19* |
| 4^{th} | 4.28 | 100% | 7 | 9 | 14 | 16 | 18 | Interferent |

Test 9

| Retention time (sec) | | | 34.1 | 44.6 | 62 | 96.2 | 111.4 | 142.9 | 188.4 | 218.6 | 384.8 | 395 | 432.6 |
|---|---|---|---|---|---|---|---|---|---|---|---|---|---|
| Compound ID | | | 4 | 5 | interferent | 8 | 9 | 11 | 12 | 14 | 16 | interferent | 18 |
| Ranking | MSR | Accuracy | Individual peak identification result | | | | | | | | | | |
| 1^{st} | 2.42 | 100% | 4 | 5 | interferent | 8 | 9 | 11 | 12 | 14 | 16 | interferent | 18 |
| 2^{nd} | 2.86 | 100% | 4 | 5 | interferent | 8 | 9 | 11 | 12 | 14 | 16 | interferent | 18 |
| 3^{rd} | 3.34 | 100% | 4 | 5 | interferent | 8 | 9 | 11 | 12 | 14 | 16 | interferent | 18 |
| 4^{th} | 5.06 | 90.9% | 4 | 5 | interferent | 8 | 10* | 11 | 12 | 14 | 16 | interferent | 18 |

TABLE S3
Test data generated from Chrom₉
Test 10

| Retention time (sec) | | | 11.92 | 21.52 | 31.2 | 40.8 | 50 | 80.8 | 108.8 | 131.6 | 173.6 | 248.8 | 393.6 |
|---|---|---|---|---|---|---|---|---|---|---|---|---|---|
| Compound ID | | | 1 | 3 | 4 | 5 | 6 | 7 | 10 | 11 | 12 | 15 | 17 |
| | | | Individual peak identification result | | | | | | | | | | |
| Ranking | MSR | Accuracy | (with experimentally generated RTT_{lib}s only) | | | | | | | | | | |
| 1^{st} | 98.99 | 81.8% | 1 | 3 | 4 | 5 | 6 | 7 | 9* | 11 | 12 | 15 | 16* |
| 2^{nd} | 99.13 | 72.7% | 1 | 2* | 4 | 5 | 6 | 7 | 9* | 11 | 12 | 15 | 16* |
| 3^{rd} | 101.48 | 81.8% | 1 | 3 | 4 | 5 | 6 | 7 | 9* | 11 | 12 | 15 | 16* |
| 4^{th} | 101.66 | 72.7% | 1 | 2* | 4 | 5 | 6 | 7 | 9* | 11 | 12 | 15 | 16* |
| | | | Individual peak identification result | | | | | | | | | | |
| Ranking | MSR | Accuracy | (with both experimentally generated and hybridized RTT_{lib}s) | | | | | | | | | | |
| 1^{st} | 1.13 | 100% | 1 | 3 | 4 | 5 | 6 | 7 | 10 | 11 | 12 | 15 | 17 |
| 2^{nd} | 2.55 | 90.9% | 1 | 2* | 4 | 5 | 6 | 7 | 10 | 11 | 12 | 15 | 17 |
| 3^{rd} | 3.16 | 90.9% | 1 | 3 | 4 | 5 | 6 | 7 | 9* | 11 | 12 | 15 | 17 |
| 4^{th} | 3.29 | 90.9% | 2* | 3 | 4 | 5 | 6 | 7 | 10 | 11 | 12 | 15 | 17 |

Test 11

| Retention time (sec) | | | 31.2 | 50.1 | 88.6 | 108.6 | 173.5 | 248.8 | 419.1 |
|---|---|---|---|---|---|---|---|---|---|
| Compound ID | | | 4 | 6 | 8 | 10 | 12 | 15 | 19 |
| | | | Individual peak identification result | | | | | | |
| Ranking | MSR | Accuracy | (with experimentally generated RTT_{lib}s only) | | | | | | |
| 1^{st} | 121.41 | 57.1% | 4 | 6 | 7* | 9* | 12 | 15 | 17* |
| 2^{nd} | 122.28 | 57.1% | 4 | 6 | 7* | 9* | 12 | 15 | 18* |
| 3^{rd} | 122.76 | 71.4% | 4 | 6 | 8 | 9* | 12 | 15 | 17* |
| 4^{th} | 123.63 | 71.4% | 4 | 6 | 8 | 9* | 12 | 15 | 18* |
| | | | Individual peak identification result | | | | | | |
| Ranking | MSR | Accuracy | (with both experimentally generated and hybridized RTT_{lib}s) | | | | | | |
| 1^{st} | 1.64 | 100% | 4 | 6 | 8 | 10 | 12 | 15 | 19 |
| 2^{nd} | 4.58 | 85.7% | 4 | 6 | 8 | 9* | 12 | 15 | 19 |
| 3^{rd} | 7.38 | 85.7% | 4 | 6 | 7* | 10 | 12 | 15 | 19 |
| 4^{th} | 7.40 | 100% | 4 | 6 | 8 | 10 | 12 | 15 | 19 |

TABLE S4 (A)

| | | | | | | | |
|---|---|---|---|---|---|---|---|
| RT in Test 5 (sec) | 87.9 | 111.4 | 218.6 | 281.4 | 384.8 | 432.6 | 494.5 |
| Compound ID | 7 | 9 | 14 | std₁ | 16 | 18 | std₂ |

Individual peak identification result w/COW aligning (slack = 1, correlation power = 1), Accuracy = 0

| | | | | | | | |
|---|---|---|---|---|---|---|---|
| RT after aligning (sec) | 109.2 | 140.2 | 265.2 | 449.5 | 497.1 | 497.1 | 575.7 |
| Peak identification | 9* | 11* | 15* | Interferent* | Interferent* | Interferent* | Interferent* |

Individual peak identification result w/COW aligning (slack = 2, correlation power = 2), Accuracy = 0

| | | | | | | | |
|---|---|---|---|---|---|---|---|
| RT after aligning (sec) | 88.2 | 115.9 | 265.2 | 345.8 | 441.8 | 499.2 | 597.3 |
| Peak identification | Interferent* | 10 | 15 | Interferent* | 19* | Interferent* | Interferent* |

Individual peak identification w/COW aligning (slack = 4, correlation power = 3), Accuracy = 57.1%

| | | | | | | | |
|---|---|---|---|---|---|---|---|
| RT after aligning (sec) | 86.2 | 109.2 | 214.4 | 281.4 | 441.8 | 490.5 | 617.6 |
| Peak identification | 7 | 9 | 14 | std₁ | 19* | std₂* | 20* |

Individual peak identification result w/COW aligning (slack = 6, correlation power = 4), Accuracy = 28.6%

| | | | | | | | |
|---|---|---|---|---|---|---|---|
| RT after aligning (sec) | 65.1 | 86.2 | 196.5 | 281.4 | 429.3 | 490.5 | 617.6 |
| Peak identification | Interferent* | 7* | 13* | std₁ | 18 | std₂* | 20* |

TABLE S4 (B)

| | | | | | | | | |
|---|---|---|---|---|---|---|---|---|
| RT in Test 7 (sec) | 87.9 | 111.4 | 218.6 | 281.4 | 340 | 384.8 | 432.6 | 494.5 |
| Compound ID | 7 | 9 | 14 | std₁ | Interferent | 16 | 18 | std₂ |

Individual peak identification result w/COW aligning (slack = 1, correlation power = 1), Accuracy = 0

| | | | | | | | | |
|---|---|---|---|---|---|---|---|---|
| RT after aligning (sec) | 109.2 | 140.2 | 265.2 | 354.1 | 429.3 | 490.5 | 545.4 | 617.6 |
| Peak identification | 9* | 11* | 15* | Interferent* | 18* | std₂ | Interferent* | 20 |

Individual peak identification result w/COW aligning (slack = 2, correlation power = 2), Accuracy = 0

| | | | | | | | | |
|---|---|---|---|---|---|---|---|---|
| RT after aligning (sec) | 88.5 | 115.9 | 265.2 | 341.9 | 425.1 | 490.5 | 545 | 617.6 |
| Peak identification | Interferent* | 10* | 15* | Interferent* | Interferent | std₂* | Interferent* | 20* |

Individual peak identification result w/COW aligning (slack = 4, correlation power = 3), Accuracy = 50%

| | | | | | | | | |
|---|---|---|---|---|---|---|---|---|
| RT after aligning (sec) | 86.2 | 109.2 | 214.4 | 275.8 | 382.1 | 441.8 | 490.5 | 617.6 |
| Peak identification | 7 | 9 | 14 | std₁ | 16* | 19* | std₂* | 20* |

Individual peak identification result w/COW aligning (slack = 6, correlation power = 4), Accuracy = 12.5%

| | | | | | | | | |
|---|---|---|---|---|---|---|---|---|
| RT after aligning (sec) | 64.7 | 86.2 | 196.5 | 271.8 | 394.7 | 441.8 | 490.5 | 617.6 |
| Peak identification | Interferent* | 7* | 13* | Interferent* | Interferent | 19* | std₂* | 20* |

TABLE S5
Test 5

| | | | | | | | |
|---|---|---|---|---|---|---|---|
| RT before aligning (sec) | 87.9 | 111.4 | 218.6 | 281.4 | 384.8 | 432.6 | 494.5 |
| Compound ID | 7 | 9 | 14 | std₁ | 16 | 18 | std₂ |

Individual peak identification result with linear warping
Same internal standards (std₁ and std₂) are adopted, Accuracy = 28.6%

| | | | | | | | |
|---|---|---|---|---|---|---|---|
| RT after aligning (sec) | 86.15 | 109.18 | 214.3 | 275.8 | 380.0 | 428.1 | 490.5 |
| Peak identification | Interferent* | Interferent* | Interferent* | std₁ | Interferent* | Interferent* | std₂ |

Test 7

| | | | | | | | | |
|---|---|---|---|---|---|---|---|---|
| RT before aligning (sec) | 87.9 | 111.4 | 218.6 | 281.4 | 340 | 384.8 | 432.6 | 494.5 |
| Compound ID | 7 | 9 | 14 | std₁ | Interferent | 16 | 18 | std₂ |

Individual peak identification result with linear warping
Same internal standards (std₁ and std₂) are adopted, Accuracy = 25%

| | | | | | | | | |
|---|---|---|---|---|---|---|---|---|
| RT after aligning (sec) | 86.315 | 109.18 | 214.3 | 275.8 | 334.84 | 380.0 | 428.1 | 490.5 |
| Peak identification | Interferent* | Interferent* | Interferent* | std₁ | Interferent* | Interferent* | Interferent* | std₂ |

TABLE S6 (A)

| | Reference | | Chrom8 | | | | Chrom9 | | | |
|---|---|---|---|---|---|---|---|---|---|---|
| Compound | | Peak | | Peak | Warped RT, | Warped RT, | | Peak | Warped RT, | Warped RT, |
| ID | RT | height | RT | height | Order = 2 | Order = 3 | RT | height | Order = 2 | Order = 3 |
| 1 | 13.9 | 17.65 | 14.1 | 18.37 | 14.14 | 14.42 | 11.9 | 2.36 | 12.47 | 11.26 |
| 2 | 19 | 15.86 | 19.2 | 12.44 | 19.11 | 19.34 | 17.3 | 7.06 | 18.27 | 17.17 |
| 3 | 23.7 | 12.73 | 23.9 | 10.50 | 23.69 | 23.88 | 21.5 | 2.52 | 22.78 | 21.76 |
| 4 | 33.6 | 16.69 | 34.1 | 12.33 | 33.63 | 33.73 | 31.2 | 7.43 | 33.19 | 32.35 |
| 5 | 43.9 | 19.63 | 44.6 | 13.72 | 43.87 | 43.90 | 40.9 | 9.08 | 43.59 | 42.91 |
| 6 | 53.6 | 18.72 | 54.6 | 13.24 | 53.63 | 53.60 | 50.1 | 8.49 | 53.44 | 52.90 |
| 7 | 86.2 | 14.21 | 87.9 | 11.26 | 86.20 | 86.02 | 80.8 | 3.90 | 86.26 | 86.07 |
| 8 | 94.4 | 16.72 | 96.2 | 12.38 | 94.33 | 94.13 | 88.6 | 6.45 | 94.58 | 94.46 |
| 9 | 109.2 | 15.85 | 111.4 | 12.11 | 109.23 | 109.00 | 102.8 | 5.20 | 109.72 | 109.69 |
| 10 | 115.9 | 14.04 | 118.2 | 11.17 | 115.90 | 115.66 | 108.6 | 3.87 | 115.90 | 115.90 |
| 11 | 140.2 | 13.45 | 142.9 | 11.12 | 140.16 | 139.93 | 131.6 | 2.74 | 140.36 | 140.45 |
| 12 | 184.8 | 15.31 | 188.4 | 11.80 | 184.98 | 184.83 | 173.5 | 4.84 | 184.80 | 184.89 |
| 13 | 196.5 | 14.65 | 200.4 | 11.54 | 196.82 | 196.71 | 184.5 | 4.29 | 196.44 | 196.50 |
| 14 | 214.4 | 17.94 | 218.6 | 13.11 | 214.81 | 214.76 | 201.4 | 6.84 | 214.29 | 214.30 |
| 15 | 265.2 | 14.18 | 270.3 | 11.43 | 266.04 | 266.22 | 248.8 | 3.80 | 264.22 | 264.02 |
| std1 | 275.8 | 14.35 | 281.4 | 11.38 | 277.06 | 277.30 | 258.6 | 3.95 | 274.52 | 274.26 |
| 16 | 382.1 | 16.02 | 384.8 | 12.06 | 380.20 | 380.88 | 367.8 | 6.01 | 388.61 | 387.89 |
| 17 | 413.4 | 14.79 | 416.6 | 11.70 | 412.08 | 412.84 | 393.7 | 4.41 | 415.50 | 414.77 |
| 18 | 429.3 | 15.38 | 432.6 | 12.09 | 428.15 | 428.93 | 407.7 | 4.56 | 430.00 | 429.30 |
| 19 | 441.8 | 15.26 | 445.4 | 12.11 | 441.02 | 441.80 | 419.1 | 4.36 | 441.80 | 441.13 |
| std2 | 490.5 | 14.00 | 494.5 | 11.64 | 490.50 | 491.20 | 464.5 | 3.36 | 488.66 | 488.31 |
| 20 | 617.6 | 13.86 | 620.3 | 11.89 | 618.09 | 617.60 | 588.1 | 2.76 | 615.21 | 617.60 |

TABLE S6 (B)
Chrom 8
Test 5

| | | | | | | | |
|---|---|---|---|---|---|---|---|
| CompoundID | 7 | 9 | 14 | std1 | 16 | 18 | std2 |
| RT | 87.9 | 111.4 | 218.6 | 281.0 | 384.6 | 432.4 | 494.3 |
| Peak height | 0.64 | 1.49 | 2.34 | 0.50 | 1.11 | 1.07 | 0.62 |
| Warped RT, | 59.10 | 86.20 | 206.67 | 274.11 | 381.95 | 429.96 | 490.50 |
| Order = 2 | | | | | | | |
| Warped RT, | 239.66 | 109.20 | 28.11 | 196.50 | 429.30 | 386.54 | 68.11 |
| Order = 3 | | | | | | | |

Test 7

| | | | | | | | | |
|---|---|---|---|---|---|---|---|---|
| CompoundID | 7 | 9 | 14 | std1 | interferent | 16 | 18 | std2 |
| RT | 87.9 | 111.4 | 218.6 | 281.0 | 340.0 | 384.6 | 432.4 | 494.3 |
| Peak height | 0.64 | 1.49 | 2.34 | 0.50 | 0.92 | 1.11 | 1.07 | 0.62 |
| Warped RT, | 59.04 | 86.20 | 206.86 | 274.33 | 336.36 | 382.10 | 430.03 | 490.41 |
| Order = 2 | | | | | | | | |
| Warped RT, | 121.10 | 86.20 | 23.70 | 52.08 | 115.90 | 184.85 | 275.80 | 415.47 |
| Order = 3 | | | | | | | | |

Chrom 9
Test 10

| | | | | | | | | | | | | | |
|---|---|---|---|---|---|---|---|---|---|---|---|---|---|
| CompoundID | 1 | 3 | 4 | 5 | 6 | 7 | 10 | 11 | 12 | 15 | std1 | 17 | std2 |
| RT | 11.9 | 21.5 | 31.2 | 40.9 | 50.1 | 80.8 | 108.6 | 131.6 | 173.5 | 248.8 | 258.6 | 393.7 | 464.5 |
| Peak height | 2.36 | 2.52 | 7.43 | 9.08 | 8.49 | 3.90 | 3.87 | 2.74 | 4.84 | 3.80 | 3.95 | 4.41 | 3.36 |
| Warped RT, | −0.46 | 10.85 | 22.25 | 33.60 | 44.33 | 79.88 | 111.73 | 137.83 | 184.80 | 267.35 | 277.91 | 419.43 | 490.50 |
| Order = 2 | | | | | | | | | | | | | |
| Warped RT, | 273.93 | 181.38 | 109.20 | 56.76 | 23.70 | 11.71 | 96.76 | 206.06 | 416.46 | 441.80 | 378.43 | −3416.86 | −8571.08 |
| Order = 3 | | | | | | | | | | | | | |

Test 11

| | | | | | | | | | |
|---|---|---|---|---|---|---|---|---|---|
| CompoundID | 4 | 6 | 8 | 10 | 12 | 15 | std1 | 19 | std2 |
| RT | 31.2 | 50.1 | 88.6 | 108.6 | 173.5 | 248.8 | 258.6 | 419.1 | 464.5 |
| Peak height | 7.43 | 8.49 | 6.45 | 3.87 | 4.84 | 3.80 | 3.95 | 4.36 | 3.36 |
| Warped RT, | 21.06 | 43.90 | 89.49 | 112.67 | 185.59 | 265.72 | 275.80 | 429.28 | 468.75 |
| Order = 2 | | | | | | | | | |
| Warped RT, | 19.00 | 43.61 | 91.84 | 115.90 | 189.44 | 266.42 | 275.80 | 410.06 | 441.80 |
| Order = 3 | | | | | | | | | |

What is claimed is:

1. A computer-implemented method for identifying compounds in a mixture, comprising:
storing a library of retention time trajectories on a non-transitory computer readable medium, where each trajectory in the library of trajectories represents a set of retention times for a group of known compounds in a mixture;
receiving, by a computer processor, a set of retention times for analytes to be identified in a sample under test;
creating, by the computer processor, a retention time trajectory for the sample under test based on correlation between retention times in the set of retention times and the group of known compounds;
comparing, by the computer processor, retention time trajectory for the sample under test to the trajectories in the library of retention time trajectories; and
identifying, by the computer processor, compounds in the sample under test based on the comparison of the retention time trajectory for the sample under test to trajectories in the library of retention time trajectories.

2. The method of claim 1 further comprises determining the set of retention times for the analytes in the sample under test using chromatography.

3. The method of claim 1 further comprises measuring a set of retention times for the analytes in the sample under test using a chromatograph and passing the set of retention times for the analytes in the sample under test to the computer processor.

4. The method of claim 1 wherein comparing the trajectory for the sample under test to trajectories in the library of trajectories further comprises calculating a similarity measure between the trajectory for the sample under test and each of the trajectories in the library of trajectories.

5. The method of claim 3 wherein the similarity measure is further defined as a mean square residual.

6. The method of claim 1 further comprises identifying presence of an interferent in the sample under test.

7. The method of claim 1 further comprises identifying presence of an interferent in the sample under test in response to a given retention time in the sample under test falling outside a tolerance for a corresponding retention time in the library of trajectories.

8. The method of claim 1 wherein creating retention time trajectories for the sample under test further comprises
enumerating possible trajectories for retention times in the set of retention times when the number of retention times in the set of retention times is less than the number of compounds in the group of known compounds, such that a given retention time in the set of retention times is mapped to only known compound and order of retention times is maintained in accordance with the group of known compounds.

9. The method of claim 1 further comprises building the library of retention time trajectories by measuring a set of retention times for the group of known analytes under different conditions using a chromatograph and thereby forming measured sets of retention time trajectories; creating new sets of retention time trajectories by linearly combining retention time trajectories from the measured sets of retention time trajectories, and adding the new sets of retention time trajectories to the library of retention time trajectories.

10. The method of claim 1 further comprises reporting the identified analytes in the sample under test visually on a display device.

11. A computer-implemented method for identifying analytes in a mixture, comprising:
storing a library of retention time trajectories on a non-transitory computer readable medium, where each trajectory in the library of trajectories represents a set of retention times for a group of known compounds;
receiving, by a computer processor, a set of retention times for analytes to be identified in a sample under test;
creating, by the computer processor, a retention time trajectory for the sample under test by pairing each retention time in the set of retention times in relation to a set of reference retention times for the group of known compounds;
comparing, by the computer processor, retention time trajectory for the sample under test to the trajectories in the library of retention time trajectories; and
identifying, by the computer processor, analytes in the sample under test based on the comparison of the retention time trajectory for the sample under test to trajectories in the library of retention time trajectories.

12. The method of claim 11 further comprises determining the set of retention times for the analytes in the sample under test using chromatography.

13. The method of claim 11 further comprises measuring a set of retention times for the analytes in the sample under test using a chromatograph and passing the set of retention times for the analytes in the sample under test to the computer processor.

14. The method of claim 11 wherein comparing the trajectory for the sample under test to trajectories in the library of trajectories further comprises calculating a similarity measure between the trajectory for the sample under test and each of the trajectories in the library of trajectories.

15. The method of claim 11 wherein creating retention time trajectories for the sample under test further comprises
enumerating possible trajectories for retention times in the set of retention times when the number of retention times in the set of retention times is less than the number of compounds in the group of known compounds, such that a given retention time in the set of retention times is mapped to only known compound and order of retention times is maintained in accordance with the group of known compounds.

16. The method of claim 11 further comprises building the library of retention time trajectories by measuring a set of retention times for the group of known analytes under different conditions using a chromatograph and thereby forming measured sets of retention time trajectories; creating new sets of retention time trajectories by linearly combining retention time trajectories from the measured sets of retention time trajectories, and adding the new sets of retention time trajectories to the library of retention time trajectories.

17. A system for identifying analytes in a mixture, comprising:
a library of retention time trajectories stored on a non-transitory computer readable medium, where each trajectory in the library of trajectories represents a set of retention times for a group of known compounds;
a chromatography configured to receive a mixture of unknown analytes and operates to output a set of retention times for analytes to be identified in the mixture; and
a computer processor interfaced with the chromatography and the library of retention time trajectories, wherein the computer processor is configured to receive the set of retention times for analytes from the chromatography and performs to:
creating, by the computer processor, a retention time trajectory for the sample under test based on correlation between retention times in the set of retention times and the group of known compounds;
comparing, by the computer processor, retention time trajectory for the sample under test to the trajectories in the library of retention time trajectories; and
identifying, by the computer processor, analytes in the sample under test based on the comparison of the retention time trajectory for the sample under test to trajectories in the library of retention time trajectories.
